SEQUENCE LISTING (18 sequences)

SEQ ID NO: 1 - DNA, 744 bp, Homo sapiens
  misc_feature: spliced 5-HT1A
atggatgtgctcagccctggtcagggcaacaacaccacatcaccaccggctccctttgagaccggcggcaacactactggtatctccgacgtgaccgtcagctaccaagtgatcacctctctgctgctgggcacgctcatcttctgcgcggtgctgggcaatgcgtgcgtggtggctgccatcgccttggagcgctccctgcagaacgtggccaattatcttattggctctttggcggtcaccgacctcatggtgtcggtgttggtgctgcccatggccgcgctgtatcaggtgctcaacaagtggacactgggccaggctgttgccaaggcaactcagcagccatttgatgtttctgcatttaatgccagttactcagattctggactctttgggatttatactatctcccaggccacagctgctggagatgttatcaaggctgcctataatcaagtaaaaacaatagctcaaggaaacctttccaacacagatgtccaagctgccaagaacaagctgaaagctggatacctaatgtcagtggagtcttctgagtgtttcctggaagaagtcgggtcccaggctctagttgctggttcttacatgccaccatccacagtccttcagcagattgattcagtggctaatgctgatatcataaatgcggcaaagaagtttgtttctggccagaagtcaatggcagcaagtggaaatttgggacatacaccttttgttgatgagttg

SEQ ID NO: 2 - protein, 248 aa, Homo sapiens
  MISC_FEATURE: spliced 5-HT1A
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQAVAKATQQPFDVSAFNASYSDSGLFGIYTISQATAAGDVIKAAYNQVKTIAQGNLSNTDVQAAKNKLKAGYLMSVESSECFLEEVGSQALVAGSYMPPSTVLQQIDSVANADIINAAKKFVSGQKSMAASGNLGHTPFVDEL

SEQ ID NO: 3 - DNA, 950 bp, Homo sapiens
  misc_feature: spliced 5-HT1A poly A
atggatgtgctcagccctggtcagggcaacaacaccacatcaccaccggctccctttgagaccggcggcaacactactggtatctccgacgtgaccgtcagctaccaagtgatcacctctctgctgctgggcacgctcatcttctgcgcggtgctgggcaatgcgtgcgtggtggctgccatcgccttggagcgctccctgcagaacgtggccaattatcttattggctctttggcggtcaccgacctcatggtgtcggtgttggtgctgcccatggccgcgctgtatcaggtgctcaacaagtggacactgggccaggctgttgccaaggcaactcagcagccatttgatgtttctgcatttaatgccagttactcagattctggactctttgggatttatactatctcccaggccacagctgctggagatgttatcaaggctgcctataatcaagtaaaaacaatagctcaaggaaacctttccaacacagatgtccaagctgccaagaacaagctgaaagctggatacctaatgtcagtggagtcttctgagtgtttcctggaagaagtcgggtcccaggctctagttgctggttcttacatgccaccatccacagtccttcagcagattgattcagtggctaatgctgatatcataaatgcggcaaagaagtttgtttctggccagaagtcaatggcagcaagtggaaatttgggacatacaccttttgttgatgagttgtaatactgatgcacacattacaggagagagctgaacgttctctcagcccagagcagcaaacacatgaaagtcagaagtctctaatatatcatttgtcttttttccagtgaggtaaaataaggcataaatgcaggtaattattcccagctgacctaaagtcaataaaacattctgtttaagtgttaaaaaaaaaaaaaaaaaaaaaa

SEQ ID NO: 4 - DNA, 931 bp, Homo sapiens
  misc_feature: spliced 5-HT1A N-term truncation
tggtcagggcaacaacaccacatcaccaccggctccctttgagaccggcggcaacactactggtatctccgacgtgaccgtcagctaccaagtgatcacctctctgctgctgggcacgctcatcttctgcgcggtgctgggcaatgcgtgcgtggtggctgccatcgccttggagcgctccctgcagaacgtggccaattatcttattggctctttggcggtcaccgacctcatggtgtcggtgttggtgctgcccatggccgcgctgtatcaggtgctcaacaagtggacactgggccagtgttgccaaggcaactcagcagccatttgatgtttctgcatttaatgccagttactcagattctggactctttgggatttatactatctcccaggccacagctgctggagatgttatcaaggctgcctataatcaagtaaaaacaatagctcaaggaaacctttccaacacagatgtccaagctgccaagaacaagctgaaagctggatacctaatgtcagtggagtcttctgagtgtttcctggaagaagtcgggtcccaggctctagttgctggttcttacatgccaccatccacagtccttcagcagattgattcagtggctaatgctgatatcataaatgcggcaaagaagtttgtttctggccagaagtcaatggcagcaagtggaaatttgggacatacaccttttgttgatgagttgtaatactgatgcacacattacaggagagagctgaacgttctctcagcccagagcagcaaacacatgaaagtcagaagtctctaatatatcatttgtcttttttccagtgaggtaaaataaggcataaatgcaggtaattattcccagctgacctaaagtcaataaaacattctgtttaagtgttaaaaaaaaaaaaaaaaaaaaaa

SEQ ID NO: 5 - DNA, 20 bp, Artificial sequence
  splice junction in spliced 5-HT1A
actgggccaggctgttgcca

SEQ ID NO: 6 - DNA, 20 bp, Artificial sequence
  forward splice primer
caggtgctcaacaagtggac

SEQ ID NO: 7 - DNA, 20 bp, Artificial sequence
  reverse splice primer
gcttggacatctgtgttgga

SEQ ID NO: 8 - DNA, 15 bp, Artificial sequence
  predicted splice site of 5-HT1A intronless
gggccaggtaacctg

SEQ ID NO: 9 - DNA, 32 bp, Artificial sequence
  oligo9F
gcgcggtcctaggcaatgcgtgcgtggtggct

SEQ ID NO: 10 - DNA, 38 bp, Artificial sequence
  oligo10R
ctcgaagcggccgcttacaactcatcaacaaaaggtgt

SEQ ID NO: 11 - DNA, 64 bp, Artificial sequence
  oligo7F
ctataggctagccaccatggactacaaggacgacgatgacaagatggatgtgctcagccctggt

SEQ ID NO: 12 - DNA, 32 bp, Artificial sequence
  oligo8R
gcattgcctaggaccgcgcagaagatgagcgt

SEQ ID NO: 13 - DNA, 20 bp, Artificial sequence
  predicted splice junction of 1A
actgggccaggtaacctgcg

SEQ ID NO: 14 - DNA, 18 bp, Artificial sequence
  splice junction
actgggccagtgttgcca

SEQ ID NO: 15 - DNA, 20 bp, Artificial sequence
  forward nucleotides 17-36
ctggtcagggcaacaacacc

SEQ ID NO: 16 - DNA, 30 bp, Artificial sequence
  reverse nucleotides 326-297
gcaacagcctggcccagtgtccacttgttg

SEQ ID NO: 17 - DNA, 20 bp, Artificial sequence
  forward nucleotides 683-702
agacggtcaaaaaggtggag

SEQ ID NO: 18 - DNA, 21 bp, Artificial sequence
  reverse nucleotides 916-897
gttgaggtttctcgtgaaacg

(19) United States
(12) Patent Application Publication
(10) Pub. No.: US 2005/0202467 A1
(43) Pub. Date: Sep. 15, 2005

(54) Novel spliced 5-HT1A receptors and methods, kits, and uses relating thereto

(75) Inventors: Bradford A. Jameson, Philadelphia, PA (US); Anna P. Tretiakova, Philadelphia, PA (US); Mark K. Haynes, Philadelphia, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Philadelphia Health and Education Corporation (d/b/a MCP Hahnemann University)

(21) Appl. No.: 11/016,991

(22) Filed: Dec. 20, 2004

Related U.S. Application Data
(60) Provisional application No. 60/531,478, filed on Dec. 19, 2003.
(60) Provisional application No. 60/541,582, filed on Feb. 4, 2004.

(51) Int. Cl. C12Q 1/68; G01N 33/567; C07H 21/04; C12N 15/09; C07K 14/72; C07K 16/28

(52) U.S. Cl. 435/6; 435/69.1; 435/320.1; 435/325; 530/388.22; 530/350; 536/23.5

(57) ABSTRACT

The present invention relates to a novel spliced 5-HT1A receptor and nucleic acids encoding the same and the polypeptides encoded thereby.

### CROSS-REFERENCE TO RELATED APPLICATIONS

The present application is entitled to priority under 35 U.S.C. § 119(e) to U.S. Provisional Application No. 60/531,478, filed on Dec. 19, 2003 and U.S. Provisional Application No. 60/541,582, filed on Feb. 4, 2004, all of which are hereby incorporated by reference in their entirety herein.

### BACKGROUND OF THE INVENTION

Serotonin (also referred to as 5-hydroxytryptamine or 5-HT) is a neurotransmitter that has been strongly implicated in the pathophysiology and treatment of a wide variety of neuropsychiatric disorders. Serotonin exerts its effects through a diverse family of serotonin receptor molecules (referred to herein as “5-HT receptors or 5-HTRs”). Classically, members of the serotonin receptor family have been grouped into subtypes according to their pharmacological specificity for various serotonin antagonists. Thus, while all the known 5-HT receptors specifically bind with serotonin, they are pharmacologically distinct and are encoded by separate genes.

To date, fourteen mammalian serotonin receptors have been identified and sequenced. These fourteen 5-HT receptors have been grouped into seven subtypes, designated 5-HTR1,5-HTR2,5-HTR3,5-HTR4,5-HTR5,5-HTR6, and 5-HTR7. Several of the subtypes are further subdivided such that the receptors are grouped pharmacologically as follows: 5-HTR1A, 5-HTR1B 5-HTR1C, 5-HTR1D, 5-HTR1E, 5-HTR1F, 5-HTR2A, 5-HTR2B, 5-HTR2C, 5-HTR3A, 5-HTR3B, 5-HTR4,5-HTR5A, 5-HTR6,5-HTR7. However, when the nucleic and amino acid sequences of the receptors are compared, the percent identity among the subtypes cannot be correlated with the pharmacological groupings.

Of the fourteen different mammalian serotonin receptors that have been cloned, all but one are members of the G-protein coupled receptor superfamily; that is, they are generally coupled to different second messenger pathways linked through guanine-nucleotide regulatory (G) proteins. For example, serotonin receptors 5-HT1A, 5-HT1B, and 5-HT1D inhibit adenylate cyclase, and 5-HT1C and 5-HT2 receptors activate phospholipase C pathways, stimulating breakdown of polyphosphoinositides. The 5-HT2 receptor belongs to the family of rhodopsin-like signal transducers which are distinguished by their seven-transmembrane configuration and their functional linkage to G-proteins.

The subtypes of serotonin receptors have been historically distinguished based on pharmacological binding profiles, on second messenger coupling, and on their physiological roles which are somewhat understood in the case of the better characterized serotonin receptors. Most of the data used to characterize 5-HT receptors is not based on the properties of a single purified receptor protein or gene, but rather, is based on experimental observations using a model tissue.

As stated previously elsewhere herein, fourteen separate serotonin receptors have been identified encompassing seven subtypes based on, inter alia, structural homology, second messenger system activation, and drug affinity for certain ligands. Molecular cloning has indicated that 5-HT receptors belong to at least two protein superfamilies: G-protein-associated receptors which have seven putative transmembrane domains (TMDs) (5-HT1A, 1B, 1C, 1D, 1E, 5-HT2), and ligand-gated ion channel receptors which have four putative TMDs (5-HT3). The 5-HT2 subfamily is further divided into 3 classes: 5-HT2A, 5-HT2B, and 5-HT2C. 5-HT2A and 5-HT2C receptor antagonists are thought to be useful in treating depression, anxiety, psychosis, and eating disorders. 5-HT2A and 5-HT2C receptors share about 51% amino acid homology overall and approximately 80% homology in their transmembrane domains.

Studies on the 5-HT2A receptor in recombinant mammalian cell lines have established that the receptor possesses two affinity states, high and low, for serotonin. Both the 5-HT2A and 5-HT2C receptors are coupled to phospholipase C and mediate responses through the phosphatidylinositol pathway. Studies using agonists and antagonists display a wide range of receptor responses suggesting that there is a wide diversity of regulatory mechanisms governing receptor activity. The 5-HT2A and 5-HT2C receptors have also been implicated as the site of action of several hallucinogenic drugs.

Serotonin is synthesized by neurons of the brain stem that project throughout the central nervous system (CNS), and highest density of serotonin is located in basal ganglia and limbic structures (Steinbusch, 1984, In: Handbook of Chemical Neuroanatomy vol. 3, pp. 68-125, Bjorklund et al., eds., Elsevier Science Publishers, B. V.). In the CNS, serotonin is believed to be involved in learning, memory, sleep, thermoregulation, motor activity, pain, sexual and aggressive behaviors, appetite, neuroendocrine regulation, and biological rhythms. Serotonin has also been linked to pathophysiological conditions such as anxiety, depression, obsessive-compulsive disorders, schizophrenia, suicide, autism, migraine, emesis, alcoholism and neurodegenerative disorders. A wide variety of sensory, motor and behavioral functions of the mammalian CNS are believed to be regulated by serotonin. Thus, understanding how 5-HT mediates its diverse physiological actions requires the identification and isolation of the pertinent 5-HT receptors.

Discrepant reports have implicated the 5-HT1A receptors but not the 5-HT2 receptors in lymphocytic stimulation (Aune et al., 1990, J. Immunol. 145:1826-1831; Aune et al., 1993, J. Immunol, 151:1175-1183; Aune et al., 1994, J. Immunol. 153:1826-1831) while other reports have implicated the 5-HT2 receptors and not 5-HT1 receptors in lymphocytic stimulation (Ameisen et al., 1989, J. Immunol. 142:3171-3179; Laberge et al., 1996, J. Immunol. 156:310-315). The research indicating 5-HT1A involvement in lymphocytic stimulation was based on using pindobind 5-HT1A as a selective receptor inhibitor, while the research indicating no 5-HT1A activity used WAY-100635 to selectively inhibit the receptor. The binding affinities of WAY-100635, p-MPPF and pindobind 5-HT1A each have a Kᵢ of less than 1 nM for the cloned human 1A receptor (Hamon et al., 1990, Neuropsychopharmacology 3:349-360; Thielen et al., 1995, Life Sci., 56:PL163-168; Liau et al., 1991, Pharmacol. Biochem. Behav., 38:555-559), while the older compound, NAN-190 has a Kᵢ of 3 nM (Boess & Martin, 1994, Neuropharmacology 33:275-317). Thus, each of these compounds has a high affinity for the 5-HT1A receptor and would be expected to inhibit receptor activity equally well. NAN-190 and pindobind 5-HT1A both reproducibly inhibited the activation of primary T cells (as well as neoplastic B and T cell lines), while WAY-100635 and p-MPPF had little or no effect on the T cell cultures at the concentrations tested, indicating that the 5-HT1A receptor is not responding to the drugs as expected for the classically defined receptor. Results describing RT-PCR attempts to amplify the 5-HT1A mRNA have also created discrepancies in the activity of this receptor. The presence of 5-HT1A mRNA in lymphocytes after mitogenic stimulation has been reported (Aune et al., 1993, J. Immunol, 151:1175-1183; Marazziti et al., 1995, Life Sci., 57:2197-2203; Abdouh et al., 2001, J. Biol. Chem., 276:4382-4388), while a separate study stated that 5-HT1A mRNA was not present in a diverse population of lymphocytes, even after mitogenic stimulation (Stefulj et al., 2000, Brain, Behavior and Immunity 14:219-224). Researchers reporting the presence of the receptor used RT-PCR primers derived from the 5′-end of the 5-HT1A message, while the negative report used primers obtained from the 3′-end.

Therefore, there exists a need to identify and characterize serotonin receptors in order to take advantage of their characteristics to diagnose and treat diseases. The present invention satisfies this need.

### BRIEF SUMMARY OF THE INVENTION

The invention relates to a novel spliced 5-HT1A receptor and nucleic acids encoding the same and the polypeptides encoded thereby.

The invention relates to an isolated nucleic acid encoding a spliced 5-HT1A receptor, and any mutants, derivatives, variants, and fragments thereof.

The invention includes an isolated nucleic acid encoding a spliced 5-HT1A receptor comprising the nucleic acid sequence set forth in SEQ ID NO:1.

In one aspect, the isolated nucleic acid shares at least about 95% identity with SEQ ID NO:1.

In yet another aspect, the isolated nucleic acid encoding a spliced 5-HT1A receptor comprises the amino acid sequence set forth in SEQ ID NO:2.

The invention also includes an isolated polypeptide comprising a spliced 5-HT1A receptor, wherein the spliced 5-HT1A receptor comprises the amino acid sequence set forth in SEQ ID NO:2.

In one aspect, the amino acid sequence shares at least about 95% identity with SEQ ID NO:2.

The invention also includes an isolated nucleic acid encoding a spliced 5-HT1A receptor comprising a nucleic acid encoding a tag polypeptide covalently linked thereto.

In one aspect, the isolated nucleic acid encoding a tag polypeptide is selected from the group consisting of a myc tag polypeptide, a glutathione-S-transferase tag polypeptide, a green fluorescent protein tag polypeptide, a myc-pyruvate kinase tag polypeptide, a His₆ tag polypeptide, an influenza virus hemagglutinin tag polypeptide, a flag tag polypeptide, and a maltose binding protein tag polypeptide.

In another aspect, the isolated nucleic acid encoding a spliced 5-HT1A receptor comprises a nucleic acid specifying a promoter/regulatory sequence operably linked thereto.

The invention includes a vector comprising an isolated nucleic acid encoding a spliced 5-HT1A receptor.

In one aspect, the vector comprises a nucleic acid specifying a promoter/regulatory sequence operably linked thereto.

The invention includes a recombinant cell comprising an isolated nucleic acid encoding a spliced 5-HT1A receptor.

In one aspect, the recombinant cell comprises a vector comprising an isolated nucleic acid encoding a spliced 5-HT1A receptor.

The invention includes an isolated nucleic acid complementary to an isolated nucleic acid encoding a spliced 5-HT1A receptor, wherein the complementary nucleic acid is in an antisense orientation to SEQ ID NO:1.

In one aspect, the recombinant cell comprising an isolated nucleic acid complementary to an isolated nucleic acid encoding a spliced 5-HT1A receptor, wherein the complementary nucleic acid is in an antisense orientation to SEQ ID NO:1.

In another aspect, the invention includes a vector comprising an isolated nucleic acid complementary to an isolated nucleic acid encoding a spliced 5-HT1A receptor, wherein the complementary nucleic acid is in an antisense orientation to SEQ ID NO:1.

The invention includes an antibody that specifically binds with a spliced 5-HT1A receptor, wherein the spliced 5-HT1A receptor comprises the amino acid sequence set forth in SEQ ID NO:2.

In one aspect, the antibody is selected from the group consisting of a polyclonal antibody, a monoclonal antibody, a humanized antibody, a chimeric antibody, and a synthetic antibody.

The invention includes a method of identifying a compound that affects binding of a spliced 5-HT1A receptor with serotonin, said method comprising contacting said receptor with a test compound and comparing the level of binding of said receptor so contacted to the level of binding in an otherwise identical receptor not contacted with said test compound, wherein a higher or lower level of receptor binding in said receptor contacted with said test compound compared to the level of receptor binding in said otherwise identical cell not contacted with said test compound is an indication that said test compound affects serotonin binding with a said receptor, thereby identifying a compound that affects binding of serotonin to said receptor.

In one aspect, a compound is identified by the method of the present invention.

In another aspect, the test compound inhibits the level of binding of serotonin with the spliced 5-HT1A receptor of the invention.

The invention includes a method of identifying a compound that affects expression of a spliced 5-HT1A receptor in a cell, said method comprising contacting a cell with a test compound and comparing the level of expression of said receptor on the cell so contacted to the level of expression of said receptor on an otherwise identical cell which is not contacted with the compound, wherein a higher or lower level of said receptor expression on said cell contacted with said test compound compared to the level of said receptor expression in said otherwise identical cell not contacted with said test compound is an indication that said test compound affects expression of said receptor on a cell, thereby identifying a compound that affects expression of said receptor.

In one aspect, the amino acid sequence of the spliced 5-HT1A receptor of the invention comprises the sequence set forth in SEQ ID NO:2.

In yet another aspect, the compound is an inhibitor selected from the group consisting of an antibody, a serotonin receptor antagonist, and a small molecule.

The invention also includes a method of identifying an isolated nucleic acid encoding a spliced 5-HT1A receptor, the method comprising amplifying said nucleic acid in a polymerase chain reaction wherein said reaction comprises a primer that specifically binds a 5′ portion of said nucleic acid, said reaction further comprising a primer that specifically binds a splice junction, the method further comprising amplifying said nucleic acid, wherein an amplification product of said reaction comprises a transchromosomal splice, thereby identifying said isolated nucleic acid comprising a transchromosomal splice.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1A depicts the nucleic acid sequence of human spliced 5-HT1A receptor isolated from normal lymphocytes (SEQ ID NO:1) (ATCC No. PTA-5792).

FIG. 1B depicts the predicted amino acid sequence of human 5-HT1A receptor (SEQ ID NO:2) encoded by the nucleic acid of FIG. 1A.

FIG. 2 is an graph depicting the response of mitogen (PHA) stimulated T-cells to selective inhibitors of the 5-HT1A receptor. Filled circles represent pindobind 5-HT1A, open circles represent WAY-100635, closed inverted triangles represent NAN-190 and open inverted triangles represent p-MPPF.

FIG. 3 is an image of a Southern blot hybridization gel depicting the detection of 5-HT1A receptor specific mRNA as a function of time after mitogen-induced proliferation of peripheral blood mononuclear cells (PBMNs). β-actin was used as an internal standard to demonstrate equal loading of the lanes.

FIG. 4 is an image comprising the cloned sequence of the spliced 5-HT1A receptor. Forward and reverse arrows refer to the primer locations for the 5-HT1A primer pair used to amplify the 5′ end of the sequence. The underlined sequences depict a splice junction.

FIG. 5, comprising FIGS. 5A and 5B is a series of images depicting a schematic diagram of the trans-splice region of the spliced 5-HT1A receptor and the translated amino acid sequence of the spliced 5-HT1A receptor. FIG. 5A depicts the 5′ open reading frame of the 5-HT1A receptor on chromosome 5 and a 3′ open reading frame from a region of chromosome 16.

FIG. 6 is an image depicting a schematic diagram of the spliced 5-HT1A receptor polypeptide. The region on the N-terminus of the splice junction comprises two transmembrane domains and two extracellular loops and the region on the carboxy terminus of the splice junction comprises a transmembrane domain directly adjacent to the splice junction and several motifs indicating cytoplasmic signal transduction domains.

FIG. 7, comprising FIGS. 7A through 7D, is a series of images depicting PCR amplification of 5-HT1A primers and primers derived from the spliced 5-HT1A receptor splice junction. FIG. 7A depicts a PCR amplification of genomic DNA using 5′ forward and reverse and 3′ forward and reverse primers to the 5-HT1A sequence and forward and reverse primers to the splice junction in CEM, Jurkat, RPMI-8226 and BE(2)-C cells. FIGS. 7B through 7D depict RT-PCR amplification using the indicated primer pairs in CEM, RPMI-8226 and BE(2)-C cells, respectively.

FIG. 8 is a schematic image depicting a plasmid vector constructed for the over-expression of spliced 5-HT1A receptor polypeptide.

FIG. 9 is a schematic image depicting a portion of the vector depicted in FIG. 8 comprising the AvrIII/NotI fragment and the NheI/AvrII fragment.

FIG. 10, comprising FIGS. 10A and 10B, is a schematic image depicting an intermediate clone of a plasmid vector comprising an isolated nucleic acid encoding a spliced 5-HT1A receptor polypeptide. FIG. 10B is a portion of the vector depicted in FIG. 10A.

FIG. 11 is a schematic image of a plasmid vector used for the transfection and expression studies described herein.

FIG. 12 is an image depicting a Western blot of transfected HeLa cells probed with an antibody to the FLAG-tag. The cells used in the Western blot were transfected with the plasmid vector indicated above each lane. pLC-2 is the plasmid vector expressing the spliced 5-HT1A receptor polypeptide.

### DETAILED DESCRIPTION OF THE INVENTION

The invention is based on the discovery of an isolated nucleic acid sequence (SEQ ID NO:1) and a protein encoded thereby (SEQ ID NO:2), designated as a spliced 5-HT1A receptor, so called because the protein comprises sequences of the 5-HT1A receptor on chromosome 5 and sequences from a region of chromosome 16.

The data disclosed herein demonstrate the isolation and characterization of a novel spliced 5-HT1A receptor present on T cells, B cells and immune cells. The invention also includes novel PCR primers for identifying novel spliced 5-HT1A receptors in a biological sample and novel methods useful for identifying novel serotonin receptors in a cell or tissue of interest.

Definitions

The articles “a” and “an” are used herein to refer to one or to more than one (i.e. to at least one) of the grammatical object of the article. By way of example, “an element” means one element or more than one element.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

“Antisense” refers particularly to the nucleic acid sequence of the non-coding strand of a double stranded DNA molecule encoding a protein, or to a sequence which is substantially identity to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

As used herein, the term “antisense oligonucleotide” means a nucleic acid polymer, at least a portion of which is complementary to a nucleic acid which is present in a normal cell or in an affected cell. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No. 5,034,506; Nielsen et al., 1991, Science 254: 1497).

“Amplification” refers to any means by which a polynucleotide sequence is copied and thus expanded into a larger number of polynucleotide molecules, e.g., by reverse transcription, polymerase chain reaction, and ligase chain reaction.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “applicator” as the term is used herein, is meant any device including, but not limited to, a hypodermic syringe, a pipette, and the like, for administering the serotonin receptor nucleic acid, protein, and/or composition of the invention to a mammal.

“Biological sample,” as that term is used herein, means a sample obtained from an animal. Preferably, the sample can be used to assess the level of expression of a serotonin receptor in the sample or the level of a serotonin receptor protein present in the sample, or both.

A “biological property” or “biological activity” of a serotonin receptor, as the term is used herein, includes, but is not limited to, the ability of the receptor to specifically bind with serotonin, to be agonized, inversely agonized, and/or antagonized, as those terms are used herein, to effect a change in the levels of cAMP in a cell, to affect the level of G-coupled protein, to transmit a signal, to affect the regulation of the immune system or the neurological system, and the like.

A “coding region” of a gene consists of the nucleotide residues of the coding strand of the gene and the nucleotides of the non-coding strand of the gene which are identity with or complementary to, respectively, the coding region of an mRNA molecule which is produced by transcription of the gene.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “disease” is a state of health of an animal wherein the animal cannot maintain homeostasis, and wherein if the disease is not ameliorated then the animal's health continues to deteriorate.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

A “genomic DNA” of a human patient is a DNA strand which has a nucleotide sequence identity with a gene of the patient. By way of example, both a fragment of a chromosome and a cDNA derived by reverse transcription of a human mRNA are genomic DNAs.

As used herein, “homology” is used synonymously with “identity”.

“Identity” as used herein, refers to nucleotide sequence similarity between two regions of the same nucleic acid strand or between regions of two different nucleic acid strands. When a nucleotide residue position in both regions is occupied by the same nucleotide residue, then the regions are identity at that position. A first region is identity to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences identity to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences identity to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the terms “gene” and “recombinant gene” refer to nucleic acid molecules comprising an open reading frame encoding a polypeptide. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins from other species (homologs), which have a nucleotide sequence which differs from that of the proteins described herein are within the scope of the invention. Nucleic acid molecules corresponding to natural allelic variants and homologs of a cDNA of the invention can be isolated based on their identity to other nucleic acid molecules using the cDNAs, or a portion thereof, as a hybridization probe according to standard hybridization techniques under stringent hybridization conditions. For example, a homolog of a human serotonin receptor protein of the invention can be isolated based on its hybridization with a nucleic acid molecule encoding all or part of a serotonin receptor under high stringency conditions.

As used herein, the term “immunogenic portion” includes any portion of a protein to which an antibody will specifically bind. For example, an immunogenic portion of a spliced 5-HT1A receptor is any portion of the polypeptide as set forth in SEQ ID NO:2 that will specifically bind with an antibody.

As used herein, an “instructional material” includes a publication, a recording, a diagram, or any other medium of expression which can be used to communicate the usefulness of the nucleic acid, peptide, and/or composition of the invention in the kit for effecting alleviation of the various diseases or disorders recited herein or for any other use encompassed herein. Optionally, or alternately, the instructional material may describe one or more methods of alleviation the diseases or disorders in a cell or a tissue of a mammal or methods of identifying or detecting a novel spliced 5-HT1A receptor in a sample, and any other use encompassed herein. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least at least about twenty sequential nucleotide-residues of the polynucleotide. It is understood that a portion of a polynucleotide may include every nucleotide residue of the polynucleotide.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

“Probe” refers to a polynucleotide that is capable of specifically hybridizing to a designated sequence of another polynucleotide. A probe specifically hybridizes to a target complementary polynucleotide, but need not reflect the exact complementary sequence of the template. In such a case, specific hybridization of the probe to the target depends on the stringency of the hybridization conditions. Probes can be labeled with, e.g., chromogenic, radioactive, or fluorescent moieties and used as detectable moieties.

A “polyadenylation sequence” is a polynucleotide sequence which directs the addition of a poly A tail onto a transcribed messenger RNA sequence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “serotonin receptor” is a receptor that specifically binds with serotonin in a sample and which does not detectably bind with any other substance or molecule in the sample.

A “receptor” is a molecule that specifically binds with a ligand.

As used herein, a “sequencing primer” is an oligonucleotide primer which is complementary to at least a portion of a polynucleotide and which can be elongated by a DNA or RNA polymerizing enzyme such as DNA polymerase, whereby binding of the sequencing primer to the polynucleotide and elongation of the primer using methods well known in the art yields an oligonucleotide transcript which is complementary to at least a part of the polynucleotide.

By the term “specifically binds,” as used herein, is meant a receptor which binds a spliced 5-HT1A receptor in a sample but does not substantially bind with any other molecule in the sample.

The term “spliced 5-HT1A receptor,” as used herein, refers to a polypeptide comprising a N-terminus region derived from a classical 5-HT1A receptor from chromosome 5 and a carboxy terminus region derived from a region of chromosome 16. A spliced 5-HT1A receptor is a chimeric protein generated as a result of a trans-splice of an open reading frame of 5-HT1A from chromosome 5 and an open reading frame from a region of chromosome 16. The 3′ end from a region of chromosome 16 is exemplified by GenBank accession No. AK094006.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis or HPLC analysis. A compound, e.g., a protein, is also substantially purified when it is essentially free of naturally associated components or when it is separated from the native contaminants which accompany it in its natural state.

A “substantially pure nucleic acid”, as used herein, refers to a nucleic acid sequence, segment, or fragment which has been purified from the sequences which flank it in a naturally occurring state, e.g., a DNA fragment which has been removed from the sequences which are normally adjacent to the fragment e.g., the sequences adjacent to the fragment in a genome in which it naturally occurs. The term also applies to nucleic acids which have been substantially purified from other components which naturally accompany the nucleic acid, e.g., RNA or DNA or proteins which naturally accompany it in the cell.

A tissue “normally comprises” a cell if one or more of the cell are present in the tissue in an animal not afflicted with a disease or disorder.

An “internal” tissue of an animal is a tissue which is normally located beneath the epidermis of the animal, within the animal's body.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

By “tag” polypeptide is meant any protein which, when linked by a peptide bond to a protein of interest, may be used to localize the protein, to purify it from a cell extract, to immobilize it for use in binding assays, or to otherwise study its biological properties and/or function.

A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### DESCRIPTION

The data disclosed herein demonstrate the discovery, cloning and expression of a novel spliced 5-HT1A receptor comprising an N-terminus region from an open reading frame of a 5-HT1A receptor from chromosome 5 and a C-terminus region comprising an open reading frame from a region of chromosome 16.

The invention relates to a novel nucleic acid encoding a spliced 5-HT1A receptor and a protein encoded thereby. This novel receptor is expressed in, inter alia, normal human lymphocytes, including T-cells, B-cells and peripheral blood mononuclear cells (PBMCs).

While the data disclosed herein demonstrates that the nucleic acids and proteins of the present invention are expressed in lymphocytes, the invention is not limited to these, or any other cells or tissues. This is because the skilled artisan, based upon the disclosure provided herein, would understand that the nucleic acids of the invention, encoding a novel spliced 5-HT1A receptor, can be expressed in other cells and tissues. Moreover, one skilled in the art when armed with the teachings provided herein would readily appreciate that homologs and variants of the novel nucleic acid of the invention may be present in other cells and tissues, and these are therefore also encompassed in the present invention.

I. Isolated Nucleic Acids

A. Sense Nucleic Acids

The present invention includes an isolated nucleic acid encoding a human spliced 5-HT1A receptor, as exemplified by SEQ ID NO:1, or a biologically active fragment thereof. In addition, the present invention also includes an isolated nucleic acid encoding a spliced 5-HT1A receptor comprising a poly A tail as set forth in SEQ ID NO:3. In yet another aspect, the invention includes an isolated nucleic acid comprising SEQ ID NO:4, which is an isolated nucleic acid encoding a spliced 5-HT1A receptor, wherein sequences from the 5′ region is missing.

The invention includes an isolated nucleic acids encoding a 5-HT1A receptor which is, preferably, at least about 90% identical to SEQ ID NO:1. More preferably, the isolated nucleic acid encoding a spliced 5-HT1A receptor is at least about 91%, more preferably, at least about 92%, more preferably, at least about 93%, more preferably, at least about 94%, more preferably, at least about 95%, more preferably, at least about 96%, more preferably, at least about 97%, more preferably, at least about 98%, and even more preferably, at least about 99% identical to SEQ ID NO:1. Most preferably, the isolated nucleic acid encoding a spliced 5-HT1A receptor is SEQ ID NO:1.

Thus, the invention also includes an isolated nucleic acid encoding a spliced 5-HT1A receptor where the nucleic acid encodes a protein which protein is preferably, at least about 90% identical to the amino acid sequence of SEQ ID NO:2. More preferably, the isolated nucleic acid encoding a spliced 5-HT1A receptor which protein is at least about 91%, more preferably, at least about 92%, more preferably, at least about 93%, more preferably, at least about 94%, more preferably, at least about 95%, more preferably, at least about 96%, more preferably, at least about 97%, more preferably, at least about 98%, and even more preferably, at least about 99% identical to SEQ ID NO:2. Most preferably, the isolated nucleic acid encodes a spliced 5-HT1A receptor having the amino acid sequence SEQ ID NO:2.

The disclosure presented herein demonstrates that the sequence of the spliced 5-HT1A receptor of the invention is present in mRNA pools from CEM cells (a CD4+ T cell line), RPMI-8226 (a multiple myeloma-derived B cell line), and peripheral blood mononuclear cells (PBMCs). It was observed that PBMCs expressed the spliced 5-HT1A receptor of the invention following mitogen-induced proliferation. The transcript of the spliced 5-HT1A receptor was constitutively present in CEM cells and RPMI-8226 cells. As such, the present invention should be construed to encompass a novel spliced 5-HT1A receptor expressed in immune cells including T cells, B cells and PBMCs. However, the invention should not be limited to the expression of the receptor solely in lymphocytes, but should be construed to include the expression of this receptor in other cell types.

The isolated nucleic acid of the invention should be construed to include an RNA or a DNA sequence encoding the spliced 5-HT1A receptor of the invention, and any modified forms thereof, including chemical modifications of the DNA or RNA which render the nucleotide sequence more stable when it is cell free or when it is associated with a cell. Chemical modifications of nucleotides may also be used to enhance the efficiency with which a nucleotide sequence is taken up by a cell or the efficiency with which it is expressed in a cell. Any and all combinations of modifications of the nucleotide sequences are contemplated in the present invention.

The skilled artisan would appreciate that, based upon the disclosure provided herein, now that the complete sequence of the nucleic acid encoding the novel receptor of the instant invention has been obtained, the sequence of the novel receptor or variants thereof expressed in other cells, tissues or organs can be readily isolated and determined using the sequences derived from the sequences set forth in SEQ ID NO:1.

The present invention should not be construed as being limited solely to the nucleic and amino acid sequences disclosed herein. Once armed with the present invention, it is readily apparent to one skilled in the art that other nucleic acids encoding spliced 5-HT1A receptors, such as those present in other species of mammals (e.g., ape, gibbon, bovine, ovine, equine, porcine, canine, feline, and the like) can be obtained by following the procedures described herein and procedures that are well-known in the art (e.g., PCR using cDNA samples) or to be developed.

Further, any number of procedures may be used for the generation of mutant, derivative or variant forms of the receptor nucleic acid of the invention using recombinant DNA methodology well known in the art such as, for example, that described in Sambrook et al. (2001, In: Molecular Cloning: A Laboratory Manual, Cold Spring Harbor Laboratory Press, New York) and Ausubel et al. (1997, In: Current Protocols in Molecular Biology, Green & Wiley, New York).

The invention includes a nucleic acid encoding a mammalian spliced 5-HT1A receptor wherein a nucleic acid encoding a tag polypeptide is covalently linked thereto. That is, the invention encompasses a recombinant nucleic acid wherein the nucleic acid encoding the tag polypeptide is covalently linked to the nucleic acid encoding the spliced 5-HT1A receptor. Such tag polypeptides are well known in the art and include, for instance, green fluorescent protein (GFP), myc, myc-pyruvate kinase (myc-PK), His₆, maltose binding protein (MBP), an influenza virus hemagglutinin tag polypeptide, a flag tag polypeptide (FLAG), and a glutathione-S-transferase (GST) tag polypeptide. However, the invention should in no way be construed to be limited to the nucleic acids encoding the above-listed tag polypeptides. Rather, any nucleic acid sequence encoding a polypeptide which may function in a manner substantially similar to these tag polypeptides should be construed to be included in the present invention.

The tagged receptor nucleic acid can be used to detect the presence of the receptor on a cell. Further, addition of a tag polypeptide facilitates isolation and purification of the “tagged” protein such that the protein of the invention can be produced and purified readily.

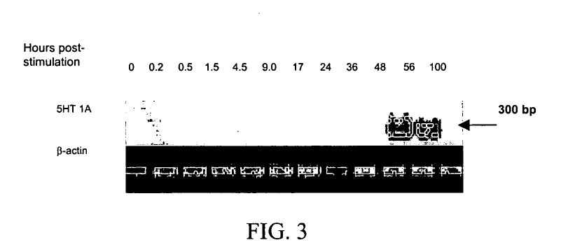

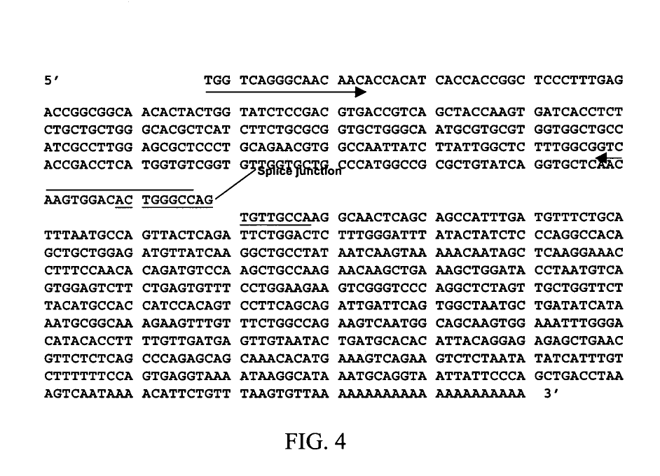

As demonstrated by the data disclosed herein, the spliced 5-HT1A nucleic acid of the present invention comprises a splice where a portion of the sequence has been mapped to chromosome 5 while another portion of the sequence was mapped to chromosome 16. The splice donor/acceptor sites in the nucleic acid encoding the novel receptor are indicated in FIG. 4. The predicted amino acid sequence (SEQ ID NO:2) of the novel receptor encoded by the nucleic acid sequence is depicted in FIG. 5B.

Modified nucleic acid sequences, i.e., nucleic acid having sequences that differ from the nucleic acid sequences encoding naturally-occurring protein, are also encompassed by the invention, so long as the modified nucleic acid still encodes a protein having the same biological activity as the spliced 5-HT1A receptor of the invention. These modifications included those caused by point mutations, modifications due to the degeneracy of the genetic code or naturally occurring allelic variants, and further modifications that have been introduced by genetic engineering, i.e., by the hand of man.

Techniques for introducing changes in nucleotide sequences that are designed to alter the functional properties of the encoded proteins or polypeptides are well known in the art. Such modifications include the deletion, insertion, or substitution of bases, and thus, changes in the amino acid sequence. Changes can be made to increase the activity of a protein, to increase its biological stability or half-life, to change its glycosylation pattern, and the like. All such modifications to the nucleotide sequences encoding such proteins are encompassed by this invention.

Further, any number of procedures may be used for the generation of mutant, derivative or variant forms of the receptor nucleic acid of the invention using recombinant DNA methodology well known in the art such as, for example, that described in Sambrook et al. (2001, In: Molecular Cloning: A Laboratory Manual, Cold Spring Harbor Laboratory Press, New York) and Ausubel et al. (1997, In: Current Protocols in Molecular Biology, Green & Wiley, New York).

Procedures for the introduction of amino acid changes in a protein or polypeptide by altering the DNA sequence encoding the polypeptide are well known in the art and are also described in Sambrook et al. (2001, supra); Ausubel et al. (1997, supra).

B. Antisense Molecules and Ribozymes

In certain situations, it may be desirable to inhibit expression of the novel spliced 5-HT1A receptor of the invention. The invention therefore includes compositions useful for inhibition of expression of the receptor of the invention. Thus, the invention includes an isolated nucleic acid complementary to a portion or all of a nucleic acid encoding the spliced 5-HT1A receptor of the invention which nucleic acid is in an antisense orientation to the receptor of the invention with respect to transcription. Preferably, the antisense nucleic acid is complementary to a nucleic acid having the sequence of SEQ ID NO:1, or a biologically active fragment thereof.

One skilled in the art will appreciate that one way to decrease the levels of receptor mRNA and/or protein in a cell is to inhibit expression of the nucleic acid encoding the protein. Expression of the receptor may be inhibited using, for example, antisense molecules, and also by using ribozymes or double-stranded RNA as described in, for example, Wianny and Kernicka-Goetz (2000, Nature Cell Biol. 2:70-75).

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue (1993, U.S. Pat. No. 5,190,931).

Alternatively, antisense molecules of the invention can be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see Cohen, supra; Tullis, 1991, U.S. Pat. No. 5,023,243, incorporated by reference herein in its entirety).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053, incorporated by reference herein in its entirety). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is that, because they are sequence-specific, only mRNAs with particular sequences are inactivated.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

Ribozymes useful for inhibiting the expression of a serotonin family receptor may be designed by incorporating target sequences into the basic ribozyme structure which are complementary to the mRNA sequence of the receptor encoded by the nucleic acid or having at least about 90% homology to SEQ ID NO:1. Ribozymes targeting the receptor may be synthesized using commercially available reagents (Applied Biosystems, Inc., Foster City, Calif.) or they may be genetically expressed from DNA encoding them.

One skilled in the art would appreciate, based upon the disclosure provided herein, that a specific transcription control sequence operably linked with a nucleic acid encoding a novel spliced 5-HT1A receptor can be a useful target for inhibition using antisense and/or ribozyme molecules. Specific inhibition of items sequence using, inter alia, antisense and/or ribozyme molecules is encompassed in the invention as would be appreciated by the skilled artisan armed with the teachings of the invention.

II. Isolated Polypeptides

The invention also includes an isolated polypeptide comprising novel human spliced 5-HT1A receptor encoded by a nucleic acid comprising the sequence of SEQ ID NO:1, or a biologically active fragment thereof. In addition, the present invention includes an isolated polypeptide comprising the sequence of SEQ ID NO:2. The receptor of the invention is present in a human lymphocyte, including T-cells, B-cells and PMBCs.

Preferably, the isolated polypeptide comprising a spliced 5-HT1A receptor is at least about 90% identical to the amino acid sequence of SEQ ID NO:2. More preferably, the isolated polypeptide is at least about 91%, more preferably, at least about 92%, more preferably, at least about 93%, more preferably, at least about 94%, more preferably, at least about 95%, more preferably, at least about 96%, more preferably, at least about 97%, more preferably, at least about 98%, and even more preferably, at least about 99% identical to SEQ ID NO:2. Most preferably, the isolated polypeptide comprising a spliced 5-HT1A receptor comprises the amino acid sequence SEQ ID NO:2.

The present invention also provides for analogs of proteins or peptides which comprise a spliced 5-HT1A receptor as disclosed herein. Analogs may differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

The present invention should also be construed to encompass “mutants,” “derivatives,” and “variants” of the peptides of the invention (or of the DNA encoding the same) which mutants, derivatives and variants are novel serotonin receptors which are altered in one or more amino acids (or, when referring to the nucleotide sequence encoding the same, are altered in one or more base pairs) such that the resulting peptide (or DNA) is not identical to the sequences recited herein, but has the same biological property as the peptides disclosed herein, in that the peptide has biological/biochemical properties of the receptor of the present invention.

Further, the invention should be construed to include naturally occurring variants or recombinantly derived mutants of the novel spliced 5-HT1A receptor sequence of the invention, which variants or mutants render the protein encoded thereby either more, less, or similarly biologically active as the full-length clones of the invention.

III. Vectors

In other related aspects, the invention includes an isolated nucleic acid encoding the novel human serotonin receptor, wherein the nucleic acids is exemplified by SEQ ID NO:1, operably linked to a nucleic acid comprising a promoter/regulatory sequence such that the nucleic acid is capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, supra), and Ausubel et al. (1997, supra).

Expression of the receptor may be accomplished by generating a plasmid, viral, or other type of vector comprising the desired nucleic acid operably linked to a promoter/regulatory sequence, which serves to drive expression of the protein in cells in which the vector is introduced, as disclosed elsewhere herein. Many promoter/regulatory sequences useful for driving constitutive expression of a gene are available in the art and include, but are not limited to, for example, the cytomegalovirus immediate early promoter enhancer sequence, the SV40 early promoter, both of which were used in the experiments disclosed herein, as well as the Rous sarcoma virus promoter, and the like. Moreover, inducible and tissue specific expression of the nucleic acid encoding the receptor of the invention may be accomplished by placing the nucleic acid encoding the receptor, with or without a tag, under the control of an inducible or tissue specific promoter/regulatory sequence. Examples of tissue specific or inducible promoter/regulatory sequences which are useful for this purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto. Expressing the receptor using a vector facilitates the isolation of large amounts of recombinantly produced protein.

Selection of any particular plasmid vector or other DNA vector is not a limiting factor in this invention and a wide plethora vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook, supra, and Ausubel, supra.

The invention thus includes a vector comprising an isolated nucleic acid encoding the novel spliced 5-HT1A receptor of the invention, exemplified herein by the spliced 5-HT1A receptor comprising the amino acid sequence as set forth in SEQ ID NO:2. The incorporation of a desired nucleic acid into a vector and the choice of vectors is well-known in the art as described in, for example, Sambrook et al., supra, and Ausubel et al., supra.

The invention also includes cells, viruses, proviruses, and the like, containing a nucleic acid encoding the novel spliced 5-HT1A receptor of the invention. The nucleic acid can be exogenously administered to a cell by a method which is well-known in the art. The nucleic acid can also be delivered to a cell, virus, or the like, by administering a vector comprising the nucleic acid to the cell, virus, or the like. Methods for producing cells comprising vectors and/or exogenous nucleic acids are well-known in the art. See, e.g., Sambrook et al., supra; Ausubel et al., supra.

A preferred vector of the invention is the plasmid designated as MH6-34 which is disclosed in more detail elsewhere herein and was deposited on Jan. 28, 2004, with the American Type Culture Collection (ATCC), Rockville, Md., and was given the ATCC Accession Number PTA-5792.

IV. Recombinant Cells

The invention includes a recombinant cell comprising, inter alia, an isolated nucleic acid encoding a spliced 5-HT1A receptor of the invention, an antisense nucleic acid complementary thereto, a nucleic acid encoding an antibody that specifically binds with the receptor of the invention, and the like. In one aspect, the recombinant cell can be transiently transfected with a plasmid encoding a portion of the nucleic acid encoding a receptor of the invention. The nucleic acid need not be integrated into the cell genome nor does it need to be expressed in the cell. Moreover, the cell may be a prokaryotic or a eukaryotic cell and the invention should not be construed to be limited to any particular cell line or cell type. Such cells include, but are not limited to, lymphocytes.

Further, the invention includes a recombinant cell comprising an antisense nucleic acid, which cell is a useful model for elucidating the role(s) of the receptor of the invention in the regulation of the immune system. That is, the inhibition of an immune response by inhibiting the receptor of the invention indicates that the serotonin-like receptor of the invention has a role in regulation of the immune system. Accordingly, a recombinant cell comprising an antisense nucleic acid complementary to all or a portion of the nucleic acid encoding the receptor of the invention is a useful tool for the study of the mechanism(s) of action of the receptor and its role(s) in the regulation of the immune system, and for the identification of therapeutics that ameliorate the effect(s) of receptor binding with serotonin and the like.

The invention includes a eukaryotic cell which, when the isolated nucleic acid of the invention is introduced therein, and the protein encoded by the nucleic acid is expressed therefrom where it was not previously present or expressed in the cell or where it is now expressed at a level or under circumstances different than that before the nucleic acid was introduced, a benefit is obtained. Such a benefit may include the fact that there has been provided a system in the expression of the nucleic acid can be studied in vitro in the laboratory or in a mammal in which the cell resides, a system wherein cells comprising the introduced nucleic acid can be used as research, diagnostic and therapeutic tools, and a system wherein animal models are generated which are useful for the development of new diagnostic and therapeutic tools for selected disease states in a e.g., autoimmune disease and allograft rejection.

Such a cell expressing an isolated nucleic acid encoding the receptor of the invention can be used to provide a novel receptor to a cell, tissue, or whole animal where a higher level of the receptor can be use to treat or alleviate a disease associated with low level of receptor expression and/or activity. Therefore, the invention includes a cell expressing the novel receptor of the invention to increase or induce receptor expression, translation, and/or activity, where increasing receptor expression, protein level, and/or activity can be useful to treat or alleviate a disease.

One skilled in the art would appreciate, based upon this disclosure, that cells comprising decreased levels of expression of the receptor of the invention, decreased level of receptor activity, or both, include, but are not limited to, cells expressing an inhibitor of receptor expression (e.g., antisense or ribozyme molecules).

The recombinant cell of the invention can be used to study the effects of elevated or decreased expression of the novel receptor of the invention on cell homeostasis and cell proliferation since antagonizing the receptor of the invention is hypothesized to play a role in regulation of the immune response, i.e., inhibition of proliferation of the cells of the immune system.

The recombinant cell of the invention, wherein the cell has been engineered such that it does not express the receptor of the invention, or expresses an altered receptor lacking biological activity, can also be used in ex vivo and in vivo cell therapies where either an animal's own cells (e.g., lymphocytes) or those of a syngeneic matched donor are recombinantly engineered as described elsewhere herein (e.g., by insertion of an antisense nucleic acid or a knock-out vector such that receptor expression and/or protein levels are thereby reduced in the recombinant cell), and the recombinant cell is administered to the recipient animal. In this way, recombinant cells that express the receptor at a reduced level can be administered to an animal whose own cells express increased levels of the receptor thereby treating or alleviating a disease, disorder or condition associated with or mediated by increased expression of the receptor of the invention as disclosed elsewhere herein.

V. Antibodies

The invention also includes an antibody that specifically binds a spliced 5-HT1A receptor of the invention, or a biologically active fragment thereof.

One skilled in the art would understand, based upon the disclosure provided herein, that the invention includes an antibody that specifically binds the receptor of the invention, such as but not limited to, the human spliced 5-HT1A receptor as set forth in SEQ ID NO:2 or an immunogenic portion thereof.

The invention should not be construed as being limited solely one type of antibody. Rather, the invention should be construed to include antibodies, as that term is defined elsewhere herein, that specifically bind to the spliced 5-HT1A receptor of the invention, or portions thereof. Further, the present invention should be construed to encompass antibodies, inter alia, that bind to the receptor of the invention and that are able to bind the receptor present on Western blots, in immunohistochemical staining of tissues thereby localizing the receptor in the tissues, and in immunofluorescence microscopy of a cell transiently transfected with a nucleic acid encoding at least a portion of the novel receptor protein.

One skilled in the art would appreciate, based upon the disclosure provided herein, that the antibody can specifically bind with any portion of the receptor of the invention and the full-length receptor can be used to generate antibodies specific therefor. However, the present invention is not limited to using the full-length receptor as an immunogen. Rather, the present invention includes using an immunogenic portion of the protein to produce an antibody that specifically binds with the spliced 5-HT1A receptor of the invention. That is, the invention includes immunizing an animal using an immunogenic portion, or antigenic determinant, of the receptor protein of the invention.

The antibodies can be produced by immunizing an animal such as, but not limited to, a rabbit or a mouse, with a protein of the invention, or a portion thereof, or by immunizing an animal using a protein comprising at least a portion of the receptor protein of the invention, or a fusion protein including a tag polypeptide portion comprising, for example, a maltose binding protein tag polypeptide portion, covalently linked with a portion comprising the appropriate receptor amino acid residues. One skilled in the art would appreciate, based upon the disclosure provided herein, that smaller fragments of these proteins can also be used to produce antibodies that specifically bind the novel receptor of the invention.

One skilled in the art would appreciate, based upon the disclosure provided herein, that various portions of an isolated polypeptide encoding the receptor of the invention can be used to generate antibodies to either highly conserved regions of receptor protein or to non-conserved regions of the polypeptide of the invention.

Further, the skilled artisan, based upon the disclosure provided herein, would appreciate that the non-conserved regions of a protein of interest can be more immunogenic than the highly conserved regions which are conserved among various organisms. Further, immunization using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of each receptor protein molecule of the invention can be used to produce antibodies that are specific only for that receptor and do not cross-react non-specifically with other serotonin receptor proteins or with other proteins.

The skilled artisan would appreciate, based upon the disclosure provided herein, that the present invention encompasses antibodies that neutralize and/or inhibit novel receptor activity (e.g., by inhibiting necessary receptor/ligand interactions).

One skilled in the art would also understand, based upon the disclosure provided herein, that it may be advantageous to inhibit the activity and/or expression of a spliced 5-HT1A receptor molecule without affecting the activity and/or expression of other serotonin receptor molecules. Thus, whether inhibition of expression and/or activity of the novel receptor is achieved using antibodies, antisense nucleic acids, and the like, one skilled in the art would appreciate, based upon the disclosure provided herein, that the present invention encompasses selectively affecting one or more receptor molecules and, in certain cases, the invention encompasses inhibiting the expression or activity of other serotonin receptors. Whether one or more serotonin receptors should be affected can be readily determined by the skilled artisan based on which disease is being treated, and the specific tissue being targeted.

The invention encompasses polyclonal, monoclonal, synthetic antibodies, and the like. One skilled in the art would understand, based upon the disclosure provided herein, that the crucial feature of the antibody of the invention is that the antibody bind specifically with the receptor of the invention. That is, the antibody of the invention recognizes the receptor of the invention, or a biologically active fragment thereof (e.g., an immunogenic portion or antigenic determinant thereof), on Western blots, in immunostaining of cells, and immunoprecipitates the receptor protein using standard methods well-known in the art.

One skilled in the art would appreciate, based upon the disclosure provided herein, that the antibody can be used to localize the relevant protein in a cell and to study the role(s) of the antigen recognized thereby in cell processes. Moreover, the antibody can be used to detect and or measure the amount of protein present in a biological sample using well-known methods such as, but not limited to, Western blotting and enzyme-linked immunosorbent assay (ELISA). The antibody can also be used to immunoprecipitate and/or immuno-affinity purify their cognate antigen using methods well-known in the art. In addition, the antibody can be used to decrease the level of novel receptor expression on a cell thereby inhibiting the effect(s) of serotonin on a cell. Thus, by administering the antibody to a cell or to the tissue of an animal, or to the animal itself, the interactions between serotonin and the receptor of the invention are therefore inhibited such that the effect of serotonin-mediated signaling are also inhibited.

Polyclonal antibodies are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the receptor portion is rendered immunogenic (e.g., receptor conjugated with keyhole limpet hemocyanin, KLH) and a portion comprising the respective amino acid residues of the human receptor of the invention. The chimeric proteins are produced by cloning the appropriate nucleic acids encoding the spliced 5-HT1A of the invention (e.g., SEQ ID NO:1, SEQ ID NO:3, SEQ ID NO:4) into a plasmid vector suitable for this purpose.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein.

Further, the antibody of the invention may be “humanized” using the technology described in, for example, Wright et al. (supra), and in the references cited therein, and in Gu et al. (1997, Thrombosis and Hematocyst 77:755-759), and other methods of humanizing antibodies well-known in the art or to be developed.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., supra.

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

In sum, one skilled in the art would appreciate, based upon the disclosure provided herein. that the antibody of the invention can be administered as a polypeptide, as a nucleic acid encoding the peptide, or both.

VI. Compositions

The invention includes a composition comprising an isolated nucleic acid complementary to a nucleic acid, or a portion thereof, encoding a spliced 5-HT1A receptor of the invention, as exemplified by SEQ ID NO:1. Preferably, the composition further comprises a pharmaceutically acceptable carrier.

The invention includes a composition comprising an isolated human spliced 5-HT1A receptor polypeptide comprising the amino acid sequence as set forth in SEQ ID NO:2 as described herein. Preferably, the composition further comprises a pharmaceutically-acceptable carrier.

VII. Methods

A. Methods of Identifying Useful Compounds

The present invention further includes a method of identifying a compound that affects binding of the spliced 5-HT1A receptor of the invention with serotonin. The method comprises contacting that receptor of the invention with a test compound and comparing the level of binding of the receptor so contacted to the level of binding of the receptor in an otherwise identical situation in the absence of the compound. If the level of binding of serotonin with the receptor of the invention in the presence of test compound is higher or lower as compared to the level of binding of serotonin with an otherwise identical receptor of the invention in the absence of test compound, this is an indication that the test compound affects binding of serotonin with the receptor of the invention.

The invention encompasses methods to identify a compound that inhibits binding of serotonin with the spliced 5-HT1A receptor of the invention but not with other 5-HT receptors. One skilled in the art would appreciate, based upon the disclosure provided herein, that assessing the level of receptor binding with serotonin can be performed using probes (e.g., antibodies and/or nucleic acid probes that specifically bind with the receptor), and or using methods to assess T cell activation otherwise mediated by such binding so that the method can identify a compound that selectively affects binding of serotonin with the receptor of the invention. Such compounds are useful for inhibiting binding of serotonin with a spliced 5-HT1A receptor while not affecting the level of binding of serotonin with another serotonin receptor family member.

One skilled in the art would understand that such compounds can be useful for inhibiting a disease modulated by and/or associated with binding of serotonin with the receptor of the invention without affecting other processes mediated by serotonin binding to other receptors which are not affected by the compound. Thus, the skilled artisan would appreciate, based on the disclosure provided herein, that it may useful to inhibit binding of serotonin with the receptor of the invention while leaving the binding of serotonin with the other serotonin receptors unaffected especially given the importance of these receptors in other processes including. but not limited to, neurological processes and immunological responses. Thus, this method is useful for identifying specific antagonists of the receptor of the invention.

The invention also encompasses methods to identify a compound that increases binding of serotonin with the spliced 5-HT1A receptor of the invention but does not affect serotonin binding with other 5-HT receptors.

The level of serotonin binding can be assessed using methods well-known in the art. Test compounds that affect receptor binding activity can be identified as follows. A radiolabeled ligand known to bind serotonin receptors can be bound to an appropriate substrate expressing one or both of these receptors. For example, radiolabeled quipazine, which is available commercially, can be used as the ligand. The compound to be tested is then incubated, along with a putative receptor, with the radiolabeled quipazine ligand combination. Displacement of radiolabeled ligand is positive evidence that the test compound being tested can affect serotonin binding with receptors. The amount of radiolabeled ligand which is displaced is determined by an appropriate standard curve which can also provide information concerning binding affinities. The displaced radiolabeled ligand can be quantitated using a standard scintillation counter.

Binding studies using ³H-quipazine are described in detail by Milburn, (1989, J. Neurochem. 52:1787-1792). Briefly, rat cortices are homogenized in 20 volumes of 50 mM Tris HCl buffer pH 7.7 at 25° C. and centrifuged at 49,000×g for 10 min. The pellet is resuspended in fresh buffer and incubated at 37° C. for 10 min. After the final centrifugation, the pellet is resuspended in 80 volumes of Krebs-HEPES buffer (25 mM HEPES, 118 mM NaCl, 5 mM KCl, 2.5 mM CaCl₂, and 1.2 mM MgCl₂ pH adjusted to 7.4). Tissue (10 mg of original wet weight) is added to assay tubes containing 0.8 nM ³H quipazine and displacing drug or buffer in a final volume of 1 ml. Non-specific binding is defined using 1 micromole zacopride. After a 30 min incubation at room temperature, the tissue is rapidly filtered under vacuum through No. 32 glass fiber filters and rinsed twice with 5 ml of 50 mM Tris-HCl buffer pH 7.7. Radioactivity is quantified by liquid scintillation counting. All experiments are performed three to six times, each in triplicate. This same approach can be used with other radiolabeled ligands such as zacopride, granisetron, haloperidol, mianserin, ketanserin, 5-HT, dopamine, droperidol, or ritanserin.

Such compounds are useful for increasing binding of serotonin with a spliced 5-HT1A receptor while not affecting the level of binding of serotonin with other serotonin receptor family members. One skilled in the art would understand that such compounds can be useful as a therapeutic for a disease modulated by and/or associated with binding of serotonin with a receptor of the invention. Thus, the skilled artisan would appreciate, based on the disclosure provided herein, that it may useful to increase binding of serotonin with the receptor of the invention while leaving the binding of serotonin with the other serotonin receptors unaffected. Thus, this method is useful for identifying specific agonists of a receptor of the invention.

One skilled in the art would understand, based upon the disclosure provided herein, that the invention encompasses identifying a compound that reduces receptor expression. Thus, the present invention includes a method of identifying a compound that reduces expression of the receptor of the invention in a cell. The method comprises contacting a cell with a test compound and comparing the level of expression of the receptor in the cell contacted with the compound to the level of expression of the receptor in an otherwise identical cell, which is not contacted with the compound. If the level of expression of the receptor is lower in the cell contacted with the compound as compared to the level in the cell that was not contacted with the compound, then that is an indication that the test compound reduces expression of the receptor in a cell.

The data disclosed herein demonstrate that there are certain immune diseases that are associated with and/or mediated by receptor expression (e.g., myasthenia gravis, idiopathic inflammatory myopathy, chronic neutropenia, rheumatoid arthritis, idiopathic thromcytopenia purpura, autoimmune hemolytic syndromes, antiphospholipid antibody syndromes, inflammatory bowel disease, Crohn's disease, ulcerative colitis, myocarditis, Guillian-Barre Syndrome, vasculitis, multiple sclerosis, neuromyelitis optica (devic's syndrome), lymphocytic hypophysitis, Graves disease, Addison's disease, hypoparathroidism, type 1 diabetes, systemic lupus erythematosus, pemphigus vulgaris, bullous pemphigoid, psoriasis, endometriosis, autoimmune orchitis, autoimmune erectile dysfunction, sarcoidosis, Wegener's granulomatosis, autoimmune deafness, Sjogren's disease, autoimmune uveoretinitis, interstitial cystitis, Goodpasture's syndrome, fibromyalgia and allogeneic graft response (i.e., any immune response directed against non-self tissue grafted into a recipient). Therefore, methods of identifying a compound that modulates by either increasing or decreasing the level of expression of the receptor of the invention, are helpful for treating and/or alleviating diseases associated with expression of the receptor, such as autoimmune diseases mediated by serotonin binding with the receptor.

The skilled artisan would appreciate, based upon the disclosure provided herein, that once armed with s nucleic acid encoding the receptor of the invention, one can readily identify a useful compound that can specifically bind with the receptor where the compound includes, but is not limited to, an antibody, a small molecule, and the like. Further, once armed with the sequences provided herein and the extensive modeling data for the serotonin receptors available to one skilled in the art, the skilled artisan can appreciate that rational design of compounds that can bind and/or affect serotonin receptor activity can be designed and assayed such that novel compounds can be identified in silico, as well as in vitro, that have the desired properties of binding with, and affecting signaling via, a spliced 5-HT1A receptor of the invention. Thus, the sequence data disclosed herein provides an important novel breakthrough for the identification of novel therapeutics based on affecting the signaling via the novel spliced 5-HT1A receptor of the invention.

The invention also includes a method of identifying a compound that increases expression of a spliced 5-HT1A receptor or a biologically active fragment thereof, on a cell. The method comprises contacting a cell with a test compound and comparing the level of expression of the receptor in the cell contacted with the compound to the level of expression of the receptor on an otherwise identical cell, which is not contacted with the compound. If the level of expression of the receptor is higher in the cell contacted with the compound compared to the level in the cell that was not contacted with the compound, then that is an indication that the test compound increases expression of the receptor on a cell.

Thus, the skilled artisan will appreciate, based upon the disclosure provided herein, that increasing expression levels of a spliced 5-HT1A receptor can be beneficial for treating a disease, disorder, or condition where increased binding of serotonin with the serotonin receptor of the invention provides a benefit. Thus, methods of identifying a compound that increases the expression level of a spliced 5-HT1A receptor can be used to treat various diseases.

One skilled in the art would appreciate, based on the disclosure provided herein, that the level of expression of the receptor in the cell can be assessed by determining the level of expression of mRNA encoding a receptor of the invention. Alternatively, the level of expression of mRNA encoding the receptor can be determined by using immunological methods to assess receptor protein production from such mRNA. Such methods include, but are not limited to, Western blot analysis using an antibody that specifically binds to a spliced 5-HT1A receptor of the invention.

Further, nucleic acid-based detection methods, such as Northern blot and PCR assays and the like, can be used as well. In addition, the level of receptor activity in a cell can also be assessed by determining the level of various parameters which can be affected by receptor activity such as, for example, the level of serotonin binding with the receptor using well-known binding assays, activation of tyrosine kinases, activation of serine/threonine kinases, activation of tyrosine phosphatases, activation of serine phosphatases, alteration of intracellular calcium fluxes, alteration in intracellular cyclic AMP levels, alteration of intracellular cyclic GMP levels and level of activation of a T cell. Thus, one skilled in the art would appreciate, based upon the extensive disclosure and reduction to practice provided herein, that there are a plethora of methods that are well-known in the art, which can be used to asses the level of expression of a spliced 5-HT1A receptor in a cell including those disclosed herein and others which may be developed in the future.

Further, one skilled in the art would appreciate based on the disclosure provided herein that, as disclosed in the examples below, a cell which lacks endogenous spliced 5-HT1A receptor expression can be transfected with a vector comprising an isolated nucleic acid encoding the receptor whereby expression of the receptor is effected in the cell. The transfected cell is then contacted with the test compound thereby allowing the determination of whether the compound affects expression of a receptor of the invention. Therefore, one skilled in the art armed with the present invention would be able to, by selectively transfecting a cell lacking detectable levels of a spliced 5-HT1A receptor using receptor-expressing vectors, identify a compound which selectively affects receptor expression, or assess the function of expressing the receptor of the invention in a cell.

B. Methods of Screening Tissue for the Presence of a Novel Spliced 5-HT1A Receptor

Methods for screening various tissues for the presence of a novel spliced 5-HT1A receptor are also encompassed by the invention.

That is, one skilled in the art would appreciate, based upon the disclosure provided herein, that various cells, and tissues comprise a spliced 5-HT1A receptor. Tissues which are included in such a screening method include any tissue which may comprise a spliced 5-HT1A receptor of the invention, including, but not limited to, tissue comprising mast cells, neural tissue, and lymph tissue.

The present invention further comprises a method of identifying a spliced 5-HT1A receptor. That is, the present invention comprises a method for identifying a polypeptide comprising a splice between chromosome 5 and chromosome 16 resulting in a novel spliced 5-HT1A receptor protein.

The method comprises obtaining an mRNA pool from a cell or tissue using techniques well known in the art and described elsewhere herein. Such methods include, but are not limited to, using TRIZOL (Invitrogen, La Jolla, Calif.), POLY(A)PURE (Ambion, Austin Tex.), and other reagents and kits available commercially. The mRNA is amplified using a primer derived from the 5′ end of the mRNA sequence of interest. As is well known in the art, a forward and reverse primer are produced for the methods of the present invention. As a non-limiting example, in order to identify a splice junction in a 5-HT1A receptor, a forward and reverse primer pair from 5-HT1A, such as a forward primer based on nucleotides 17-36 (ctgg tcagggcaacaacacc; SEQ ID NO:15) and a reverse primer based on nucleotides 326-297 (gcaacagcctggcccagtgtccacttgttg; SEQ ID NO:16). The skilled artisan is aware of methods for producing primers for other known sequences. The method further comprises preparing primers based on a splice junction, such as the splice junction disclosed herein (SEQ ID NO:5). As a non-limiting example, primers specific for a splice junction, including SEQ ID NO:6 and SEQ ID NO:7, are used in an amplification reaction, such as the reaction disclosed herein, to identify a novel spliced 5-HT1A receptor polypeptide comprising a trans-chromosomal splice junction.

VIII. Pharmaceutical Compositions

The invention further includes a composition comprising an isolated and purified spliced 5-HT1A receptor wherein the composition further comprises a pharmaceutically-acceptable carrier. Such a composition can be used to immunize a mammal in order to generate antibodies that specifically bind a spliced 5-HT1A receptor. Further, the composition can be used to administer a spliced 5-HT1A receptor to a mammal.

For administration to of the above-mentioned compositions to a mammal, a polypeptide, or the nucleic acid encoding it, or both, can be suspended in any pharmaceutically-acceptable carrier, for example, HEPES buffered saline at a pH of about 7.8. Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer a spliced 5-HT1A receptor.

In addition, any compound identified using any of the methods described herein can be formulated and administered to a mammal.

VIII. Kits

The invention includes various kits which comprise a compound, such as a nucleic acid encoding a novel spliced 5-HT1A receptor, an antibody that specifically binds such a receptor, a nucleic acid complementary to a nucleic acid encoding such a receptor but in an antisense orientation with respect to transcription, and/or compositions of the invention, an applicator, and instructional materials which describe use of the compound to perform the methods of the invention. Although exemplary kits are described below, the contents of other useful kits will be apparent to the skilled artisan in light of the present disclosure. Each of these kits is included within the invention.

The kit further includes a pharmaceutically-acceptable carrier. The composition is provided in an appropriate amount as set forth elsewhere herein.

The skilled artisan would appreciate, based upon the disclosure provided herein, that the invention encompasses kits where ribozymes, antibodies that specifically bind with a novel spliced 5-HT1A receptor, and the like, are comprised to reduce the level of receptor expression.

Further, the invention comprises kits comprising a nucleic acid encoding the novel spliced 5-HT1A receptor. Such kits can be used according to the methods of the invention wherever increased receptor expression is desired. That is, where a disease, disorder, or condition is associated with or mediated by decreased level of receptor compared with normal non-disease level of receptor, the kit can be used pursuant to the teachings disclosed elsewhere herein, to provide the receptor to a cell wherein the level of receptor in the cell is less than the level of receptor in an otherwise identical but normal (i.e., not diseased) cell and/or to an animal comprising such a cell.

In another aspect, the invention comprises kits for screening various tissues for detection of the novel spliced 5-HT1A receptor. Such kits comprise at least one of SEQ ID NOS:6 and 7 primers and the instructions for use thereof.

The invention also encompasses kits for identifying a compound that affects expression of a novel spliced 5-HT1A receptor. The kit further comprises an applicator and an instructional material for the use thereof.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

The experiments presented in this example may be summarized as follows.

The data disclosed herein demonstrate the identification and characterization of a novel spliced 5-HT1A receptor. Further, the data demonstrate novel methods using various PCR primer designs to isolate a novel spliced 5-HT1A receptor from a cell, tissue, or organ of interest since, as demonstrated herein.

Further, the disclosure provided herein provides methods for identifying novel serotonin receptors in a tissue of interest. That is, the data disclosed herein demonstrate a novel strategy and PCR primers useful for identification of a novel spliced 5-HT1A receptor in immune system cells. The methods disclosed herein can be readily applied to any tissue to identify a novel spliced 5-HT1A receptor.

The Materials and Methods used in the experiments presented in this example are now described.

Identification of Novel 5-HT1A Receptor

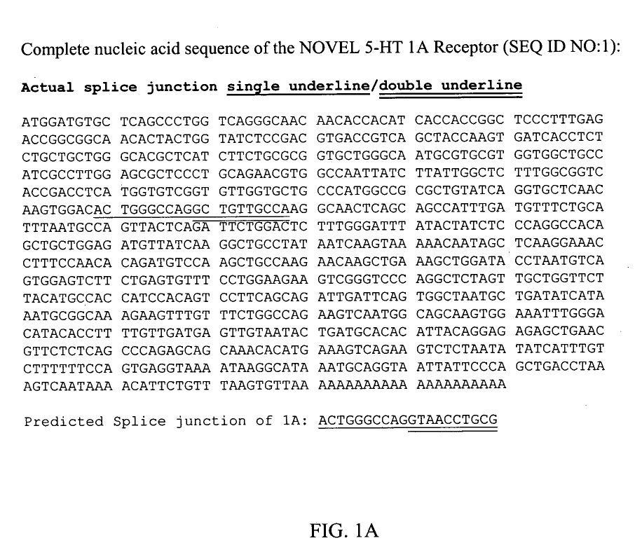

A novel nucleic acid encoding a serotonin 1A receptor was identified. A nucleic acid encoding the receptor was cloned into a plasmid designated MH6-34. The sequence encoding the full-length 5HT1A receptor was obtained and is set forth in FIG. 1 and is designated as SEQ ID NO:1. Further, the predicted amino acid sequence of the polypeptide encoded by the novel nucleic acid is set forth at FIG. 1B and is designated as SEQ ID NO:2. Due to cloning methodology used, the nucleic acid contained in plasmid MH6-34 does not encode approximately the first amino acid residues of the sequence set forth in FIG. 1B (SEQ ID NO:2). The sequence of these amino acid residues was determined according to standard recombinant methods well known in the art and therefore not discussed herein.

Deposit of Biological Materials

The following exemplary materials have been deposited on Jan. 28, 2004, with the American Type Culture Collection (ATCC), Rockville, Md., and designated as indicated. That is, a plasmid designated MH6-34 was deposited with the ATCC and was given the ATCC Accession Number PTA-5792.

This deposit will be maintained under the terms of the Budapest Treaty on the International Recognition of the Deposit of Micro-organisms for purposes of patent procedures. More specifically, such terms include that the ATCC is a depository afforded permanence of the deposit and ready accessibility thereto by the public if a patent is granted. All restrictions on the availability to the public of the material so deposited will be irrevocably removed upon granting of a patent. The material will be readily available during the pendency of the patent application to one determined by the Commissioner to be entitled thereto under 37 C.F.R. § 1.14 and 35 U.S.C. § 122. The deposited material will be maintained with all the care necessary to keep it viable and uncontaminated for a period of at least five years after the most recent request for the furnishing of a sample of the deposited material, and in any case, for a period of at least thirty (30) years after the date of the deposit or for the enforceable life of the patent, whichever period is longer. The duty to replace the deposit should the depository be unable to furnish a sample when requested due to the condition of the deposit is hereby acknowledged.

This deposit is provided merely as convenience to those of skill in the art, and is not an admission that a deposit is required under 35 U.S.C. § 112. The nucleic acid sequence of this plasmid, as well as the amino add sequence of the polypeptide encoded thereby, are incorporated herein by reference and are controlling in the event of any conflict with the description herein. A license may be required to make, use, or sell the deposited materials, and no such license is hereby granted.

5-HT1A Binding Assays

Discrepant reports have implicated the 5-HT1A receptors but not the 5-HT2 receptors in lymphocytic stimulation (Aune et al., 1990, J. Immunol. 145:1826-1831; Aune et al., 1993, J. Immunol, 151:1175-1183; Aune et al., 1994, J. Immunol. 153:1826-1831) while other reports have implicated the 5-HT2 receptors and not 5-HT1 receptors in lymphocytic stimulation (Ameisen et al., 1989, J. Immunol. 142:3171-3179; Laberge et al., 1996, J. Immunol. 156:310-315). Research indicating 5-HT1A involvement in lymphocytic stimulation was based on using pindobind 5-HT1A as a selective receptor inhibitor, while the research indicating no 5-HT1A activity used WAY-100635 to selectively inhibit the receptor. The binding affinities of WAY-100635, p-MPPF and pindobind 5-HT1A each have a Kᵢ of less than 1 nM for the cloned human 1A receptor (Hamon et al., 1990, Neuropsychopharmacology 3:349-360; Thielen et al., 1995, Life Sci., 56:PL163-168; Liau et al., 1991, Pharmacol. Biochem. Behav., 38:555-559), while NAN-190 has a Kᵢ of 3 nM (Boess & Martin, 1994, Neuropharmacology 33:275-317). Thus, each of these compounds has a high affinity for the 5-HT1A receptor and would be expected to inhibit receptor activity equally well. NAN-190 and pindobind 5-HT1A both reproducibly inhibited the activation of primary T cells (as well as neoplastic B and T cell lines), while WAY-100635 and p-MPPF had little or no effects on the T cell cultures at the concentrations tested, indicating that the 5-HT1A receptor is not responding to the drugs as expected for the classically defined receptor.

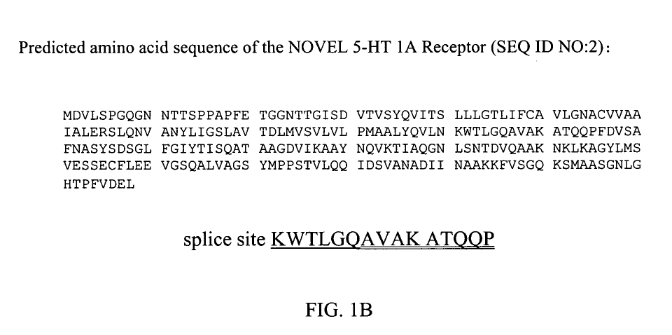

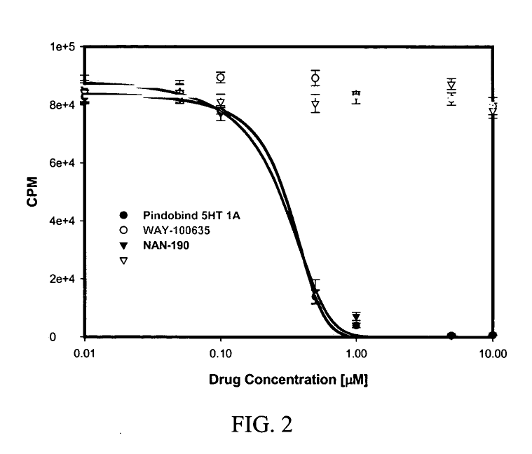

Functional pharmacology studies were employed to evaluate four different and highly selective inhibitors of the 5-HT1A receptors using methods disclosed herein. Each of the tested compounds has a high affinity for the 5-HT1A receptor and was expected to inhibit the receptor's activity equally well. The results of these experiments demonstrated that NAN-190 and pindobind 5-HT1A both reproducibly inhibited the activation of primary T cells, neoplastic B and T cell lines, while WAY-100635 and p-MPPF had little or no effects on the T cell cultures at the concentrations tested (FIG. 2). These data indicate that the 5-HT1A receptor is not responding to the drugs as would be expected for the ‘classically’ defined receptor.

RT-PCR Amplification of Spliced 5-HT1A Receptor

Results describing RT-PCR attempts to amplify the 5-HT1A receptor mRNA have also created discrepancies in the activity of this receptor. The presence of 5-HT1A mRNA in lymphocytes after mitogenic stimulation has been reported (Aune et al., 1993, J. Immunol, 151:1175-1183; Marazziti et al., 1995, Life Sci., 57:2197-2203; Abdouh et al., 2001, J. Biol. Chem., 276:4382-4388), while a separate study stated that 5-HT1A mRNA was not present in a diverse population of lymphocytes, even after mitogenic stimulation (Stefulj et al., 2000, Brain, Behavior and Immunity 14:219-224). Researchers reporting the presence of the receptor used RT-PCR primers derived from the 5′-end of the 5-HT1A message, while the negative report used primers obtained from the 3′-end.

Human RT-PCR primers derived from the 5′ end of the 5-HT1A sequence were used to amplify a band corresponding to the 5-HT1A receptor at 48 hours post mitogenic stimulation. All PCR reactions described herein were performed using high fidelity HF-2 polymerase at 20 cycles of PCR to reduce mutagenesis. The 300 base pair amplification product was sequenced and found to be collinear with the known 5-HT1A sequence. A wide variety of primers derived from the 3′-end of the 5-HT1A sequence were designed and synthesized, but none of the primer pairs amplified 5-HT1A cDNA. An analysis of the intronless 5-HT1A genomic sequence, revealed a potential donor splice site. A variety of different splice site prediction algorithms were employed, all of which predicted a donor splice site with a high degree of confidence (greater than 80%). The splice site prediction obtained from the “Splice Site Prediction by Neural Network” (Berkeley Drosophila Genome Project) indicated a splice site with a score of 0.85 between nucleotides 312 and 326 of the intronless 5-HT1A genomic sequence with a sequence of gggccagGTaacctg (SEQ ID NO:8; capital letters indicate exon/intron boundary).

Isolation and Sequencing of Spliced 5-HT1A Receptor

The pharmacological and RT-PCR anomalies and the presence of a potential donor splice site prompted a reexamination of the mRNA encoding the 5-HT1A receptor using a rapid amplification of cDNA ends (RACE) method, which is known in the art. A clonal B cell population (RPMI-8226, ATCC, Manassas, Va.) was used to avoid the heterogeneity associated with PBMCs. The forward primer was the same 5′ primer used in previous RT-PCR experiments (indicated by the forward arrow in FIG. 4). The reverse primer was a unique sequence tag attached to the poly-A tail of the mRNA. The intervening sequence of the cDNA was amplified via PCR and cloned into a vector supplied by the manufacturer. The vector was amplified in bacteria and sequenced. The 300 base pair RT-PCR product was ³²P-labeled and used as a probe to screen the cloned RACE-PCR products in a Southern blot. All of the clones that hybridized to this probe were sequenced. The sequence of the cloned product is depicted in FIG. 4.

All of the sequence 5′ of the predicted splice site was identical to the intronless open reading frame of human chromosome 5 that encodes the 5-HT1A receptor. All of the sequence that is 3′ of the predicted splice site is identical to an open reading frame found on chromosome 16 (GenBank Acc. No. AK094006), a putative protein identified as a part of the human genome sequencing efforts. This unknown protein bears homology to the ubiquinol-cytochrome c reductase complex core protein 2 precursor (the mitochondrial targeting signal of this protein was not present in the spliced 5-HT1A sequence). The sequenced transcript represents a novel transchromosomal splice between chromosome 5 and chromosome 16 of the human genome. The spliced sequence encodes an uninterrupted amino acid sequence depicted in FIG. 5B. Every clone derived from the RACE-PCR that recognized the 300 bp probe comprised this sequence.

Bioinformatics Analysis of Spliced 5-HT1A

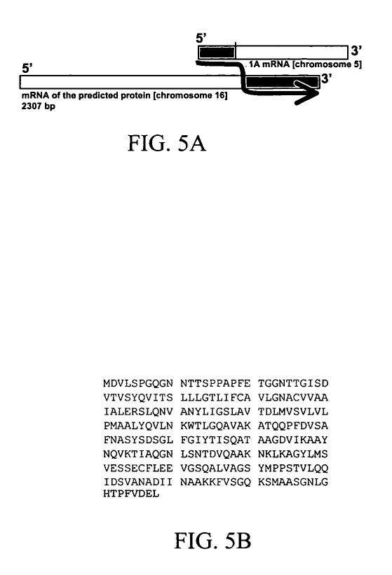

A bioinformatics analysis of the chimeric protein indicated that the protein section spliced to the 5-HT1A sequence has a potential transmembrane domain and several potential cytoplasmic signal transduction domains (FIG. 6). The spliced 5-HT1A receptor amino acid sequence encoded by the trans-spliced nucleic acid encompasses the first two transmembrane domains and the first two extracellular loops of the 5-HT1A receptor and is spliced to the carboxy terminal domain of the open reading frame found on chromosome 16.

Spliced 5-HT1A Receptor in Multiple Cell Lines

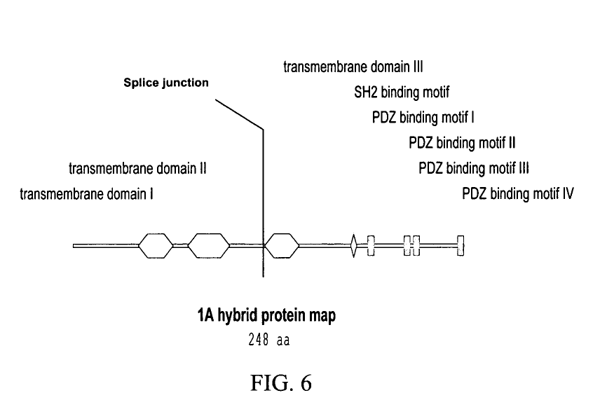

Reports have demonstrated the existence of trans-splicing in lower eukaryotes and in mammalian systems (Zhan et al., 2003, J. Immunol. 165:3612-3619; Garcia-Blanco, 2003, J. Clin. Invest. 112: 474-480). To confirm that the spliced 5-HT1A receptor is indeed a trans-splice and not an artifact of PCR amplification, several different cell lines were probed for the presence of the spliced 5-HT1A receptor transcript. The 5′ primers were derived from the 5-HT1A sequence (forward nucleotides 17-36 (ctgg tcagggcaacaacacc; SEQ ID NO:15) and reverse nucleotides 326-297 (gcaacagcctggcccagtgtccacttgttg; SEQ ID NO:16) (300 base pair fragment)). The three prime primers were derived from the 5-HT1A sequence (forward nucleotides 683-702 (agacggtcaaaaaggtggag; SEQ ID NO:17) and reverse nucleotides 916-897 (gttgaggtttctcgtgaaacg; SEQ ID NO:18) (234 base pair fragment)). The splice primers were: forward caggtgctcaacaagtggac (SEQ ID NO:6) and reverse gcttggacatctgtgttgga (SEQ ID NO:7). FIG. 7 demonstrates that the splice junction cannot be amplified from genomic DNA samples. RT-PCR was used to determine whether the novel splice event was present in the mRNA pools from CEM cells (a CD4+ T cell line), BE(2)-C (a neuroblastoma cell line), and RPMI-8226 (a multiple myeloma-derived B cell line). The 5′-end of the mRNA was amplified in all cell lines, however, the 3′-end could only be amplified in the neuroblastoma line. The data demonstrate the presence of the splice junction in both the CEM and the RPMI-8226 cells, but no splice junction was detectable in the neuroblastoma cells. These data demonstrate that the splice junction is present in the tested immune derived cells.

Verification of the Spliced 5-HT1A mRNA

To further confirm the presence of the spliced 5-HT1A receptor transcript, mRNA from RPMI-8226 cells and PHA-stimulated (20 μg/ml, 48 hour) CEM cells was isolated and RT-PCR reactions were performed as described herein. The splice junction primers were used in this amplification (forward, 5′-CAG GTG CTC AAC AAG TGG AC-3′ (SEQ ID NO:6); corresponding to positions 288 to 308 of the human 5-HT1A receptor gene and reverse 5′-GCT TGG ACA TCT GTG TTG GA-3′(SEQ ID NO:7); corresponding to position 379 to 399 of the core protein II of the human cytochrome bcl complex). The appropriately sized PCR products were cut from the agarose gels and isolated using a gel isolation kit from Qiagen (Valencia, Calif.) according to the manufacturer's instructions. Sequencing of the gel-purified products revealed that the obtained sequences contained the expected splice junction.

Construction of a Spliced 5-HT1A Expression Vector

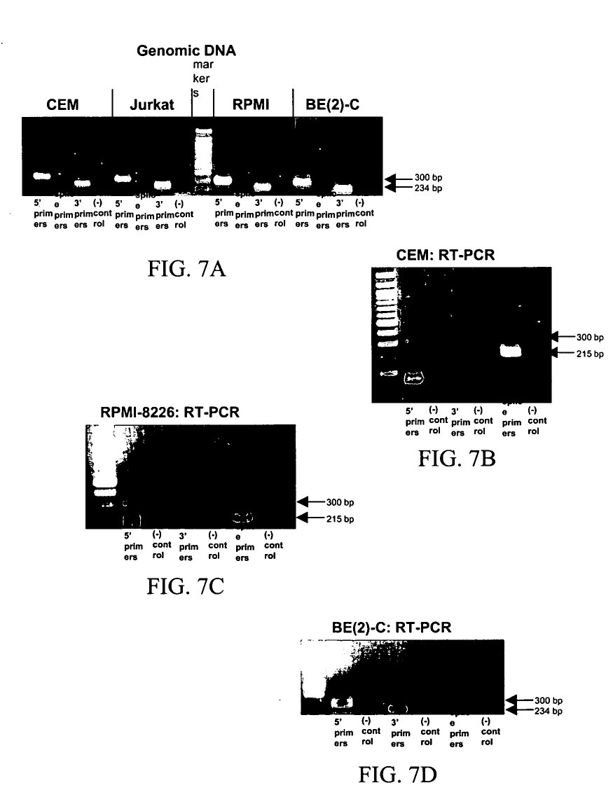

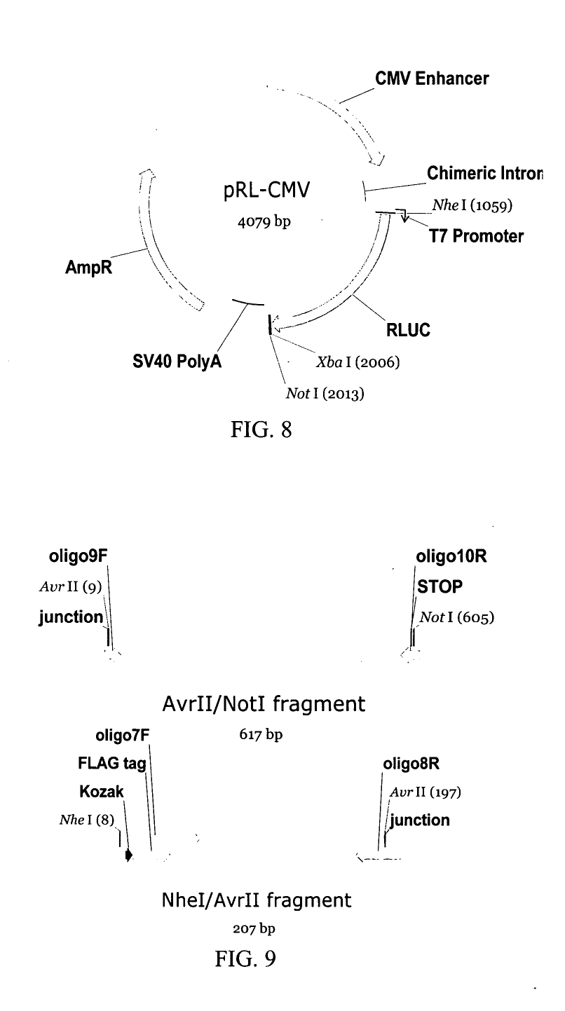

The vector used for constructing an over-expression system for the spliced 5-HT1A receptor is depicted in FIG. 8. To construct the vector, the pRL-CMV vector (Promega, Madison, Wis.) was digested with NheI/NotI to eliminate the coding sequence of Renilla Luciferase [RL] and linearize the vector.

The cloned spliced 5-HT1A nucleic acid derived from the RACE-PCR studies contained the full length transcript minus a small part of the 5′-terminal end. This sequence was amplified using the forward primer containing an AvrII adapter (oligo9F: gcgcggtcctaggcaatgcgtgcgtggtggct (SEQ ID NO:9)) and the reverse primer containing a terminator-NotI adapter (oligo10R: ctcgaagcggccgcttacaactcatcaacaaaaggtgt (SEQ ID NO:10)) to produce the AvrII/NotI fragment. The resulting fragment was gel purified according to methods described elsewhere herein.

The missing N-terminal region of the 5-HT1A receptor was amplified from an mRNA library derived from RPMI-8226 cells using the forward primer containing a NheI-Kozak-FLAG adapter (oligo7F: ctataggctagccaccatggactacaaggacgacgatgacaa gatggatgtgctcagccctggt (SEQ ID NO:11)) and a reverse primer containing an AvrII adapter (oligo8R: gcattgcctaggaccgcgcagaagatgagcgt (SEQ ID NO:12) to produce the NheI/AvrII fragment. This fragment was also gel purified.

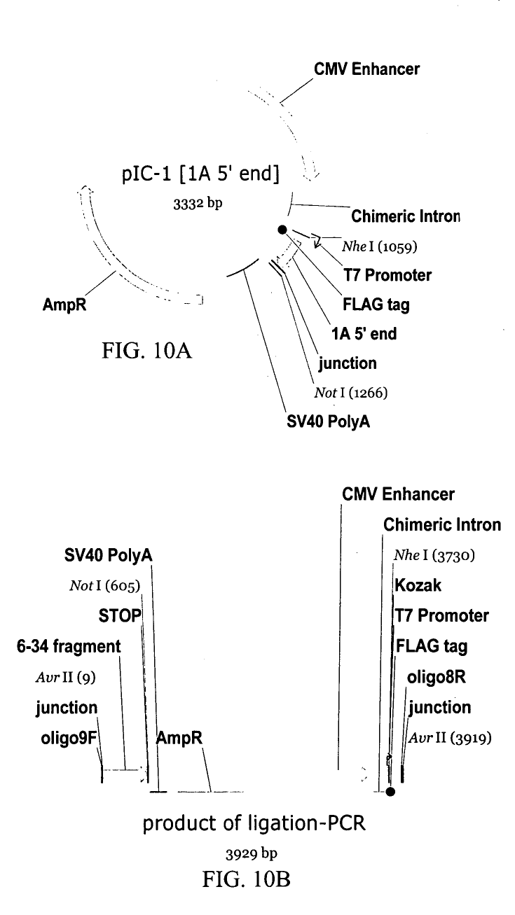

Next, the NheI/AvrII fragment was cloned into a calf intestinal alkaline phosphatase (CIP) treated NheI/XbaI-digested vector. The resulting clones were screened for proper orientation and the intermediate clones were sequenced (pIC-1 plasmid, FIG. 10. While the AvrII and XbaI cuts have compatible CTAG overhangs, ligation of the AvrII site into the XbaI site destroys both sites.

To clone the intact full-length spliced 5-HT1A receptor, the pIC-1 vector was digested with NotI and the CIP-treated digested vector was ligated the AvrII/NotI fragment into the NotI site. The product of the ligation was used as a template to PCR-amplify the entire vector using the reverse primer containing AvrII adapter (SEQ ID NO:12) and forward primer containing AvrII adapter SEQ ID NO:9).

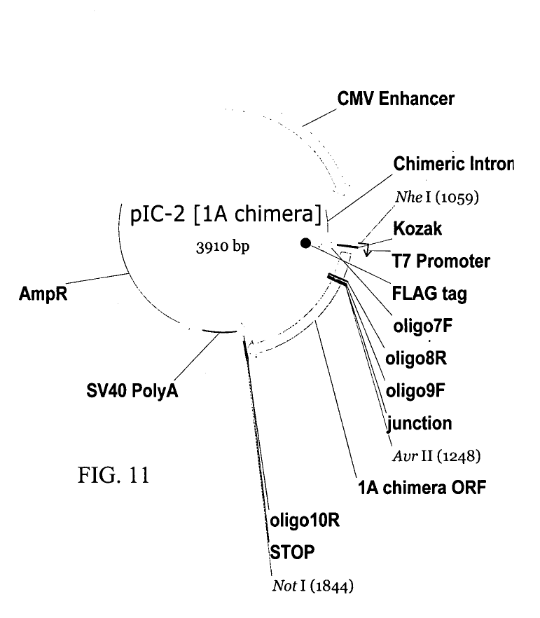

The PCR product was digested with DpnI to remove the methylated template and was subsequently gel-purified. The gel-purified product was digested with AvrII and self-ligated to create the pIC-2 plasmid (FIG. 11). The final pIC-2 plasmid was sequenced to ensure that no point mutations or other aberrations were introduced.

Transfection and Expression Studies

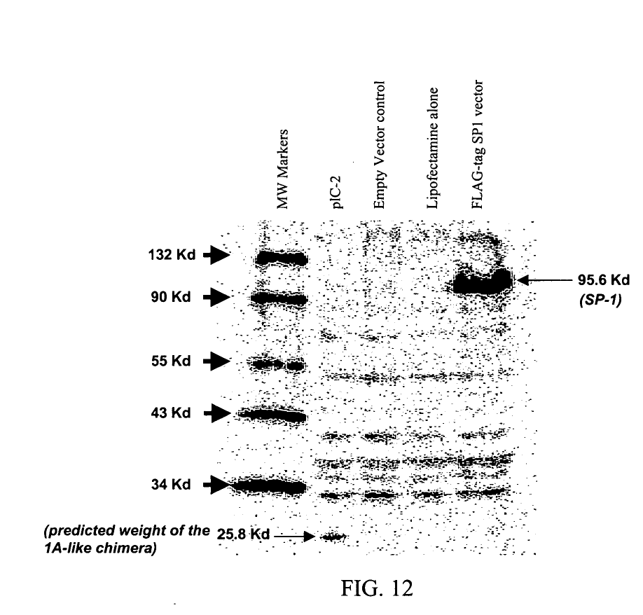

The pIC-2 plasmid was transfected into HeLa cells using Lipofectamine-2000 (Invitrogen, La Jolla Calif.). An expression vector containing green fluorescent protein and one containing a FLAG-tagged SP1 protein was used as controls in these studies. Transfection efficiency was optimized and based on the percentage of cells expressing the green fluorescent protein, the estimated transfection efficiency at 48 hours post-transfection was between 75-80% of the HeLa cells. The FLAG-tagged proteins were detected by Western blot analysis 48 hours post-transfection. Radioimmunoprecipitation (RIPA) cell lysis buffer containing a protease inhibitor cocktail was added to the HeLa cell monolayers and the resultant lysate was collected, passed through a 21 gauge needle, incubated on ice for 1 hour and centrifuged. Boiled cell lysates from transfected and non-transfected cultures were electrophoresed in 10% acrylamide and transferred to nitrocellulose. Blots containing separated proteins were blocked (BLOTTO Western blot blocking buffer) and probed with an anti-FLAG antibody at a dilution of 1/1000 followed by detection with an HRP-conjugated secondary antibody. FLAG-containing proteins were visualized by chemiluminescence (FIG. 12).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An isolated nucleic acid encoding a human spliced 5-HT1A receptor, wherein said isolated nucleic acid comprises the nucleic acid sequence set forth in SEQ ID NO:1.

2. An isolated nucleic acid encoding a human spliced 5-HT1A receptor, wherein the amino acid sequence of said human spliced 5-HT1A receptor comprises the amino acid sequence set forth in SEQ ID NO:2.

3. An isolated polypeptide comprising a human spliced 5-HT1A receptor, wherein said spliced 5-HT1A receptor comprises the amino acid sequence set forth in SEQ ID NO:2.

4. The isolated nucleic acid of claim 1, said nucleic acid further comprising a nucleic acid encoding a tag polypeptide covalently linked thereto.

5. The isolated nucleic acid of claim 4, wherein said tag polypeptide is selected from the group consisting of a myc tag polypeptide, a glutathione-S-transferase tag polypeptide, a green fluorescent protein tag polypeptide, a myc-pyruvate kinase tag polypeptide, a His₆ tag polypeptide, an influenza virus hemagglutinin tag polypeptide, a flag tag polypeptide, and a maltose binding protein tag polypeptide.

6. The isolated nucleic acid of claim 1, said nucleic acid further comprising a nucleic acid specifying a promoter/regulatory sequence operably linked thereto.

7. A vector comprising the isolated nucleic acid of claim 1.

8. The vector of claim 7, said vector further comprising a nucleic acid specifying a promoter/regulatory sequence operably linked thereto.

9. A recombinant cell comprising the isolated nucleic acid of claim 1.

10. A recombinant cell comprising the vector of claim 7.

11. An isolated nucleic acid complementary to an isolated nucleic acid encoding a spliced 5-HT1A receptor, said complementary nucleic acid being in an antisense orientation to SEQ ID NO:1.

12. A recombinant cell comprising the isolated nucleic acid of claim 11.

13. A vector comprising the isolated nucleic acid of claim 11.

14. An antibody that specifically binds with the spliced 5-HT1A receptor, wherein said spliced 5-HT1A receptor comprises the amino acid sequence set forth in SEQ ID NO:2.

15. The antibody of claim 14, wherein said antibody is selected from the group consisting of a polyclonal antibody, a monoclonal antibody, a humanized antibody, a chimeric antibody, and a synthetic antibody.

16. A method of identifying a compound that affects binding of a spliced 5-HT1A receptor with serotonin, said method comprising contacting said receptor with a test compound and comparing the level of binding of said receptor so contacted to the level of binding in an otherwise identical receptor not contacted with said test compound, wherein a higher or lower level of receptor binding in said receptor contacted with said test compound compared to the level of receptor binding in said otherwise identical cell not contacted with said test compound is an indication that said test compound affects serotonin binding with a said receptor, thereby identifying a compound that affects binding of serotonin to said receptor.

17. A compound identified by the method of claim 16.

18. The method of claim 16, wherein said test compound inhibits the level of binding of serotonin with said receptor.

19. A method of identifying a compound that affects expression of a spliced 5-HT1A receptor in a cell, said method comprising contacting a cell with a test compound and comparing the level of expression of said receptor on the cell so contacted to the level of expression of said receptor on an otherwise identical cell which is not contacted with the compound, wherein a higher or lower level of said receptor expression on said cell contacted with said test compound compared to the level of said receptor expression in said otherwise identical cell not contacted with said test compound is an indication that said test compound affects expression of said receptor on a cell, thereby identifying a compound that affects expression of said receptor.

20. The method of claim 19, wherein the amino acid sequence of said receptor comprises the sequence set forth in SEQ ID NO:2.

21. The method of claim 19, wherein said compound is an inhibitor selected from the group consisting of an antibody, a serotonin receptor antagonist, and a small molecule.

22. A method of identifying an isolated nucleic acid encoding a spliced 5-HT1A receptor, the method comprising amplifying said nucleic acid in a polymerase chain reaction wherein said reaction comprises a primer that specifically binds a 5′ portion of said nucleic acid, said reaction further comprising a primer that specifically binds a splice junction, the method further comprising amplifying said nucleic acid, wherein an amplification product of said reaction comprises a transchromosomal splice, thereby identifying said isolated nucleic acid comprising a transchromosomal splice.
